(19) United States
(12) Patent Application Publication
(10) Pub. No.: US 2011/0223093 A1
(43) Pub. Date: Sep. 15, 2011

(54) IONIC LIQUID SYSTEMS FOR CARBON DIOXIDE AND SULFUR DIOXIDE REMOVAL

(75) Inventor: MARK A. SCIALDONE, West Grove, PA (US)

(73) Assignee: E.I. DU PONT DE NEMOURS AND COMAPNY, Wilmington, DE (US)

(21) Appl. No.: 13/045,820

(22) Filed: Mar. 11, 2011

Related U.S. Application Data
(60) Provisional application No. 61/313,181, filed on Mar. 12, 2010.

(51) Int. Cl. C01B 31/20; C07D 231/12; C07D 403/06; C07D 233/58; C07D 213/53; C07D 401/06; C07D 237/08; C07D 239/26; C07D 241/12; C07C 335/08; C07F 9/28; C01B 17/50

(52) U.S. Cl. 423/437.1; 548/375.1; 548/312.4; 548/306.1; 548/338.5; 548/314.4; 548/255; 548/267.6; 548/266.6; 548/266.4; 548/253; 548/247; 548/236; 548/214; 548/204; 546/331; 546/274.4; 546/273.7; 544/224; 544/238; 544/335; 544/333; 544/336; 544/405; 546/175; 546/146; 548/362.5; 548/261; 564/31; 564/15; 548/112; 548/113; 423/539

(57) ABSTRACT

This invention relates to sulfur functionalized ionic liquid compounds that are useful in methods of carbon dioxide or sulfur dioxide removal to which they may be applied.

This application claims priority under 35 U.S.C. §119(e) from, and claims the benefit of, U.S. Provisional Application No. 61/313,181, filed Mar. 12, 2010, which is by this reference incorporated in its entirety as a part hereof for all purposes.

### TECHNICAL FIELD

This invention relates to compounds useful in the removal of carbon dioxide and/or sulfur dioxide from a gaseous mixture, and to compositions produced thereby.

### BACKGROUND

There is increasing interest in methods to reduce or capture CO₂ from many different gaseous mixtures. CO₂ is an undesired component that is present in many gas streams such as natural gas and effluent gases, and there is also much global interest in reducing CO₂ emissions from combustion exhaust for the prevention of global warming. CO₂ can be removed or captured by many means, such as physical or chemical absorption of the gas by a liquid or solid.

Currently, a common method of carbon dioxide capture from process streams in industrial complexes involves the use of aqueous solutions of alkanolamines, but usually on a small scale. The process has been used commercially since the early 1930s (see, for example, Kohl and Nielsen, Gas Purification, 5th Edition, Gulf Publishing, Houston Tex., 1997), and is based on the reaction of a weak base (alkanolamine) with a weak acid (CO₂) to produce a water-soluble salt. This reaction is reversible, and the equilibrium is temperature dependent.

The use of alkanolamines as absorbents for CO₂ (from power plant flue gases, for example) is somewhat disadvantaged in respect of the amount of energy needed to regenerate the CO₂-rich solvent, the size of the CO₂ capture plant, and the loss of alkanolamines to the environment. Among conventional alkanolamines, monoethanolamine (MEA) is considered an attractive solvent at low partial pressures of CO₂ because it reacts at a rapid rate and the cost of the raw materials is low compared to that of secondary and tertiary amines. The costs of absorption processes using MEA are high, however, because of the high energy consumption in regeneration, and because of operation problems such as corrosion, solvent loss and solvent degradation. Furthermore, MEA can be loaded up to only 0.5 mol of CO₂/mol of MEA, or 33 mol %, as a result of the stable carbonates formed.

Physical absorption systems have advantages over chemical absorption such as lower energy costs, but also have disadvantages such as solvent losses and low CO₂ capacity. A need thus remains for systems and materials capable of providing low-cost, high-capacity methods of CO₂ capture. Concurrently, there is also interest in methods to reduce or capture SO₂ from many different gaseous mixtures. Ideally the same process and compounds could be used for both gases, with the capability to selectively release the gases upon demand.

### SUMMARY

Provided is an ionic compound as represented by the structure of the following Formula I, Formula II, Formula III, or Formula IV:

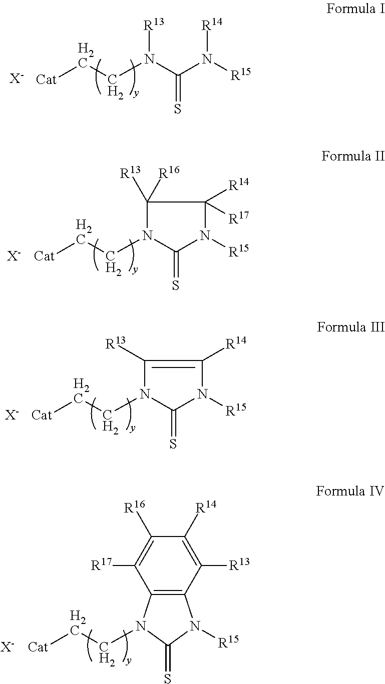

wherein y is a value in the range of 0 to 15 inclusive; and R¹³, R¹⁴, R¹⁵, R¹⁶, and R¹⁷ is each independently selected from the group consisting of:

(a) H,

(b) a —CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH;

(c) a —CH₃, —C₂H₅, or —C₃ to C₂₅ straight-chain, branched or cyclic alkane or alkene group comprising one to three heteroatoms selected from the group consisting of O, N, Si and S, and optionally substituted with at least one member selected from the group consisting of Cl, Br, F, I, OH, NH₂ and SH;

(d) a C₆ to C₂₀ unsubstituted aryl group, or a C₆ to C₂₅ unsubstituted heteroaryl group having one to three heteroatoms independently selected from the group consisting of O, N, Si and S;

(e) a C₆ to C₂₅ substituted aryl group, or a C₆ to C₂₅ substituted heteroaryl group having one to three heteroatoms independently selected from the group consisting of O, N, Si and S; wherein the substituted aryl or substituted heteroaryl group has one to three substituents independently selected from the group consisting of:
(i) a —CH₃, —C₂H5, or —C₃ to C₂₅ straight-chain, branched or cyclic alkane or alkene group, optionally substituted with at least one member selected from the group consisting of Cl, Br, F, I, OH, NH₂ and SH,
(ii) OH,
(iii) NH₂, and
(iv) SH; and

(f) —(CH₂)ₙSi(CH₂)ₘCH₃, —(CH₂)ₙSi(CH₃)₃, or —(CH₂)ₙOSi(CH₃)ₘ, where each n is independently a value in the range of 1 to 4 inclusive, and each m is independently a value in the range of 0 to 4; and

wherein any of R¹³, R¹⁴, R¹⁵, R¹⁶, and R¹⁷ can together form a ring;

wherein X⁻ is an anion; and Cat is a cation that is in each instance independently selected from the group of cations as follows:

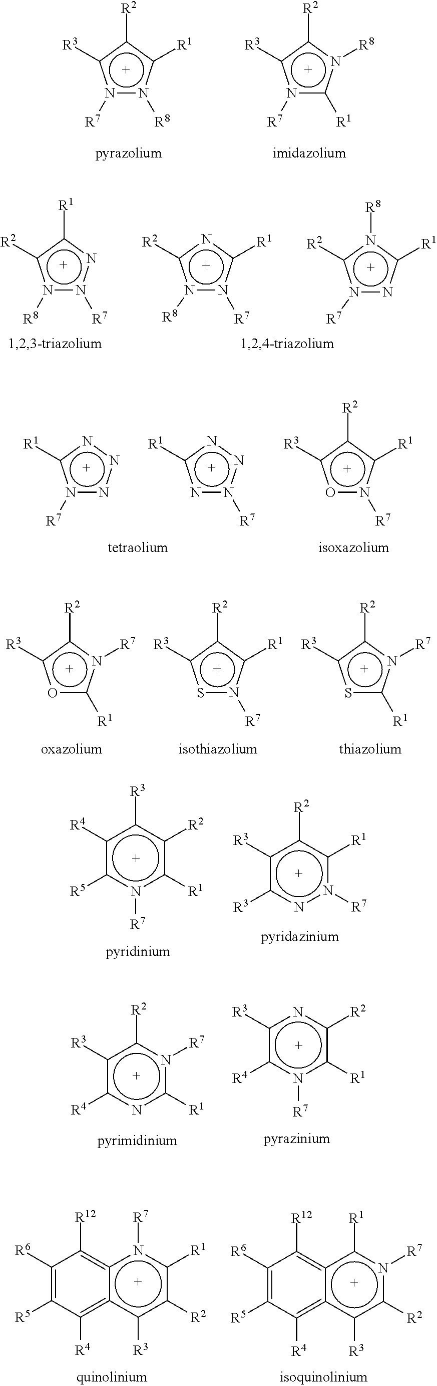

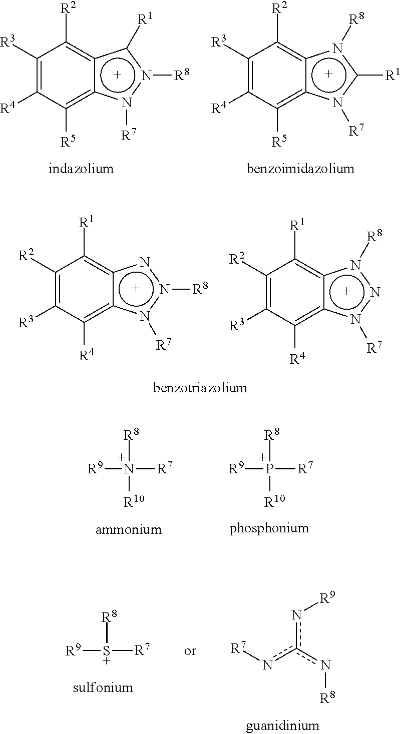

wherein in Cat (according to the formula for each of the members in the group of cations set forth above):

(a) R¹, R², R³, R⁴, R⁵, R⁶, and R¹² are independently selected from the group consisting of:

(i) H,

(ii) halogen,

(iii) a —CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH;

(iv) a —CH₃, —C₂H₅, or —C₃ to C₂₅ straight-chain, branched or cyclic alkane or alkene group comprising one to three heteroatoms selected from the group consisting of O, N, Si and S, and optionally substituted with at least one member selected from the group consisting of Cl, Br, F, I, OH, NH₂ and SH;

(v) a C₆ to C₂₀ unsubstituted aryl group, or a C₆ to C₂₅ unsubstituted heteroaryl group having one to three heteroatoms independently selected from the group consisting of O, N, Si and S;

(vi) a C₆ to C₂₅ substituted aryl group, or a C₆ to C₂₅ substituted heteroaryl group having one to three heteroatoms independently selected from the group consisting of O, N, Si and S; and wherein said substituted aryl or substituted heteroaryl group has one to three substituents independently selected from the group consisting of:
(A) a —CH₃, —C₂H₅, or C₃ to C₂₅ straight-chain, branched or cyclic alkane or alkene group, optionally substituted with at least one member selected from the group consisting of Cl, Br, F, I, OH, NH₂ and SH,
(B) OH,
(C) NH₂, and
(D) SH;

(vii) —(CH₂)ₙSi(CH₂)ₘCH₃, —(CH₂)ₙSi(CH₃)₃, or —(CH₂)ₙOSi(CH₃)ₘ, wherein each n is independently a value in the range of 1 to 4 and each m is independently a value in the range of 0 to 4;

(b) R⁷, R⁸, R⁹, and R¹⁰ is each independently selected from the group consisting of:

(i) a —CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH;

(ii) a —CH₃, —C₂H₅, or —C₃ to C₂₅ straight-chain, branched or cyclic alkane or alkene group comprising one to three heteroatoms selected from the group consisting of O, N, Si and S, and optionally substituted with at least one member selected from the group consisting of Cl, Br, F, I, OH, NH₂ and SH;

(iii) a C₆ to C₂₅ unsubstituted aryl group, or a C₆ to C₂₅ unsubstituted heteroaryl group having one to three heteroatoms independently selected from the group consisting of O, N, Si and S; and

(iv) a C₆ to C₂₅ substituted aryl group, or C₆ to C₂₅ substituted heteroaryl group having one to three heteroatoms independently selected from the group consisting of O, N, Si and S; and wherein said substituted aryl or substituted heteroaryl has one to three substituents independently selected from the group consisting of:
(A) a—CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH,
(B) OH,
(C) NH₂, and
(D) SH;

(iv) —(CH₂)ₙSi(CH₂)ₘCH₃, —(CH₂)ₙSi(CH₃)₃, or —(CH₂)ₙOSi(CH₃)ₘ, where each n is independently a value in the range of 1 to 4, and each m is independently a value in the range of 0 to 4; and (c) optionally at least two of R¹, R², R³, R⁴, R⁵, R⁶, R⁷, R⁸, R⁹, and R¹⁰ can together form a cyclic or bicyclic alkanyl or alkenyl group.

The above described compounds are useful for the purpose of capturing CO₂ and/or SO₂ from a gaseous stream.

Also provided is a composition as represented by the structure of the following Formula IA, Formula IIA, Formula IIIA, or Formula IVA:

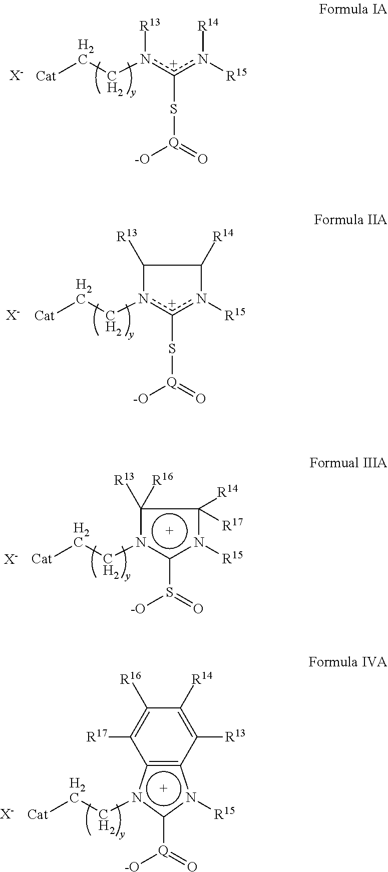

wherein Q is C or S; y is a value in the range of 0 to 15 inclusive; and R¹³, R¹⁴, R¹⁵, R¹⁶, and R¹⁷ is each independently selected from the group consisting of:

(a) H,

(b) a —CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH;

(c) a —CH₃, —C₂H₅, or —C₃ to C₂₅ straight-chain, branched or cyclic alkane or alkene group comprising one to three heteroatoms selected from the group consisting of O, N, Si and S, and optionally substituted with at least one member selected from the group consisting of Cl, Br, F, I, OH, NH₂ and SH;

(d) a C₆ to C₂₀ unsubstituted aryl group, or a C₆ to C₂₅ unsubstituted heteroaryl group having one to three heteroatoms independently selected from the group consisting of O, N, Si and S;

(e) a C₆ to C₂₅ substituted aryl group, or a C₆ to C₂₅ substituted heteroaryl group having one to three heteroatoms independently selected from the group consisting of O, N, Si and S; wherein the substituted aryl or substituted heteroaryl group has one to three substituents independently selected from the group consisting of:
(i) a —CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH,
(ii) OH,
(iii) NH₂, and
(iv) SH; and

(f) —(CH₂)ₙSi(CH₂)ₘCH₃, —(CH₂)ₙSi(CH₃)3, or —(CH₂)ₙOSi(CH₃)ₘ, where each n is independently a value in the range of 1 to 4 inclusive, and each m is independently a value in the range of 0 to 4; and

wherein any of R¹³, R¹⁴, R¹⁵, R¹⁶, and R¹⁷ can together form a ring;

wherein X⁻ is an anion; and Cat is a cation that is in each instance independently selected from the group of cations as follows:

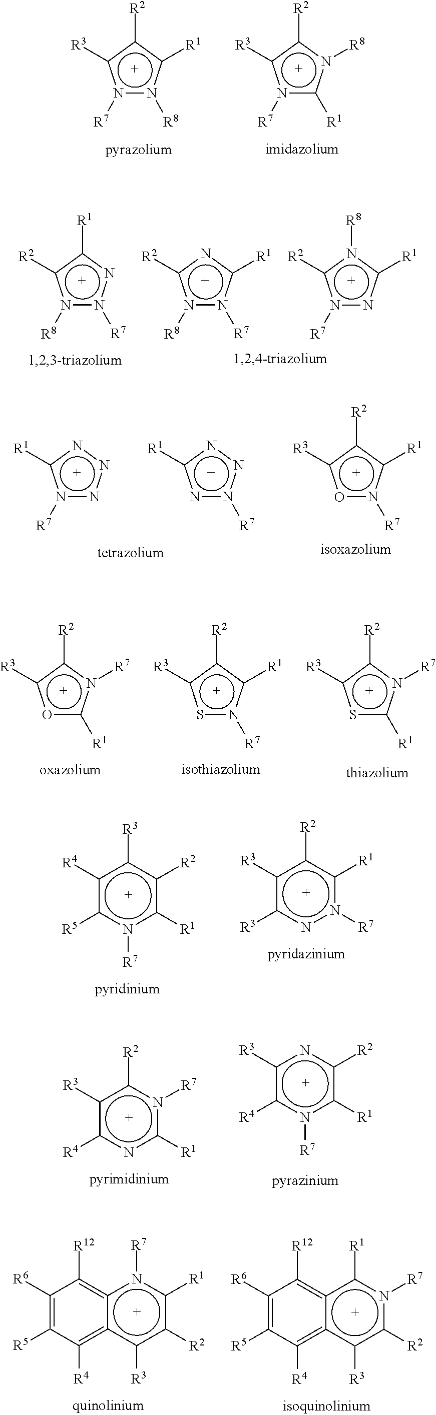

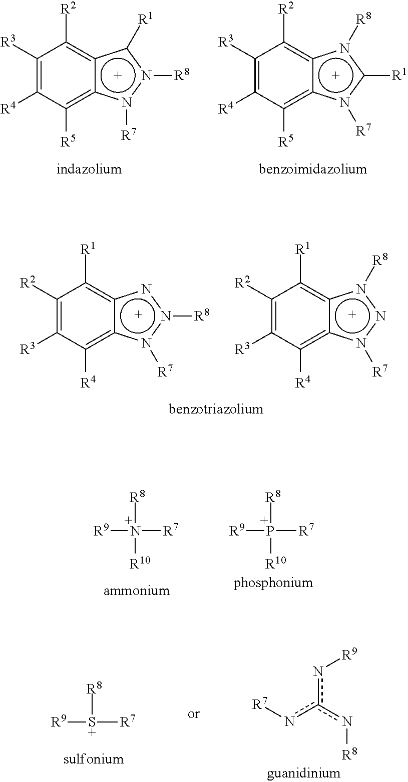

wherein in Cat (according to the formula for each of the members in the group of cations set forth above):

(a) R¹, R², R³, R⁴, R⁵, R⁶, and R¹² are independently selected from the group consisting of:

(i) H,

(ii) halogen,

(iii) a —CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH;

(iv) a —CH₃, —C₂H₅, or —C₃ to C₂₅ straight-chain, branched or cyclic alkane or alkene group comprising one to three heteroatoms selected from the group consisting of O, N, Si and S, and optionally substituted with at least one member selected from the group consisting of Cl, Br, F, I, OH, NH₂ and SH;

(v) a C₆ to C₂₀ unsubstituted aryl group, or a C₆ to C₂₅ unsubstituted heteroaryl group having one to three heteroatoms independently selected from the group consisting of O, N, Si and S;

(vi) a C₆ to C₂₅ substituted aryl group, or a C₆ to C₂₅ substituted heteroaryl group having one to three heteroatoms independently selected from the group consisting of O, N, Si and S; and wherein said substituted aryl or substituted heteroaryl group has one to three substituents independently selected from the group consisting of:
(A) a —CH₃, —C₂H₅, or C₃ to C₂₅ straight-chain, branched or cyclic alkane or alkene group, optionally substituted with at least one member selected from the group consisting of Cl, Br, F, I, OH, NH₂ and SH,
(B) OH,
(C) NH₂, and
(D) SH;

(vii) —(CH₂)ₙSi(CH₂)ₘCH₃, —(CH₂)ₙSi(CH₃)₃, or —(CH₂)ₙOSi(CH₃)ₘ, wherein each n is independently a value in the range of 1 to 4 and each m is independently a value in the range of 0 to 4;

(b) R⁷, R⁸, R⁹, and R¹⁰ is each independently selected from the group consisting of:

(i) a —CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH;

(ii) a —CH₃, —C₂H₅, or —C₃ to C₂₅ straight-chain, branched or cyclic alkane or alkene group comprising one to three heteroatoms selected from the group consisting of O, N, Si and S, and optionally substituted with at least one member selected from the group consisting of Cl, Br, F, I, OH, NH₂ and SH;

(iii) a C₆ to C₂₅ unsubstituted aryl group, or a C₆ to C₂₅ unsubstituted heteroaryl group having one to three heteroatoms independently selected from the group consisting of O, N, Si and S; and

(iv) a C₆ to C₂₅ substituted aryl group, or C₆ to C₂₅ substituted heteroaryl group having one to three heteroatoms independently selected from the group consisting of O, N, Si and S; and wherein said substituted aryl or substituted heteroaryl has one to three substituents independently selected from the group consisting of:
(A) a—CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH,
(B) OH,
(C) NH₂, and
(D) SH;

(iv) (—CH₂)ₙSi(CH₂)ₘCH₃, —(CH₂)ₙSi(CH₃)₃, or —(CH₂)ₙOSi(CH₃)ₘ, where each n is independently a value in the range of 1 to 4, and each m is independently a value in the range of 0 to 4; and

(c) optionally at least two of R¹, R², R³, R⁴, R⁵, R⁶, R⁷, R⁸, R⁹, and R¹⁰ can together form a cyclic or bicyclic alkanyl or alkenyl group.

The above described compositions are useful for the purpose of providing a source material from which can be obtained either the compounds of Formulae I˜IV, and/or CO₂ and/or SO₂.

This invention also provides a method of generating an ionic compound as represented by the structure of the Formula I, II, III or IV, or a mixture thereof, by heating a composition as represented by the structure of the Formula IA, IIA, IIIA or IVA, or mixture thereof, and/or by contacting the composition as represented by the structure of the Formula IA, IIA, IIIA or IVA, or mixture thereof, with a non-solvent, and recovering the compound as represented by the structure of the Formula I, II, III or IV, or a mixture thereof.

This invention also provides a method of generating CO₂ and/or SO₂ by heating a composition as represented by the structure of the Formula IA, IIA, IIIA or IVA, or mixture thereof, and/or by contacting the composition as represented by the structure of the Formula IA, IIA, IIIA or IVA, or mixture thereof, with a non-solvent, and recovering CO₂ and/or SO₂.

### BRIEF DESCRIPTION OF THE DRAWING

FIG. 1 is a drawing of a process that can be used to selectively absorb and release SO₂ and CO₂ from a gaseous mixture.

### DETAILED DESCRIPTION

In the description of the several embodiments of the inventions hereof, the following definitional structure is provided for certain terminology as employed variously in the specification:

An “alkyl” group is a monovalent (i.e. having a valence of one) group represented by the formula CₙH₂ₙ₊₁.

An “aryl” group is a monovalent radical formed by removal of a hydrogen atom from a hydrocarbon that is structurally composed entirely of one or more benzene rings.

A “heteroaryl” refers to unsaturated rings of 6 or more atoms containing one or two O and S atoms and/or one to four N atoms provided that the total number of hetero atoms in the ring is 4 or less, or bicyclic rings wherein a five or six membered ring containing O, S, and N atoms as defined above is fused to a benzene or pyridyl ring.

In one embodiment hereof, there are provided one or more of the compounds represented by Formula I, Formula II, Formula III, or Formula IV:

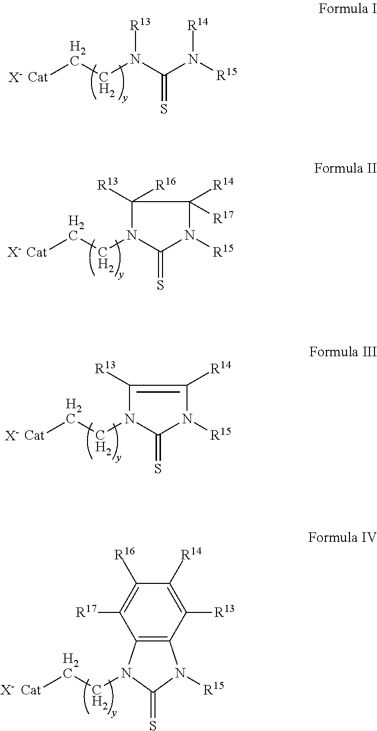

wherein y is a value in the range of 0 to 15 inclusive; and R¹³, R¹⁴, R¹⁵, R¹⁶, and R¹⁷ is each independently selected from the group consisting of:

(a) H,

(b) a —CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH;

(c) a —CH₃, —C₂H₅, or —C₃ to C₂₅ straight-chain, branched or cyclic alkane or alkene group comprising one to three heteroatoms selected from the group consisting of O, N, Si and S, and optionally substituted with at least one member selected from the group consisting of Cl, Br, F, I, OH, NH₂ and SH;

(d) a C₆ to C₂₀ unsubstituted aryl group, or a C₆ to C₂₅ unsubstituted heteroaryl group having one to three heteroatoms independently selected from the group consisting of O, N, Si and S;

(e) a C₆ to C₂₅ substituted aryl group, or a C₆ to C₂₅ substituted heteroaryl group having one to three heteroatoms independently selected from the group consisting of O, N, Si and S; wherein the substituted aryl or substituted heteroaryl group has one to three substituents independently selected from the group consisting of:
(i) a —CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH,
(ii) OH,
(iii) NH₂, and
(iv) SH; and

(f) —(CH₂)ₙSi(CH₂)ₘCH₃, —(CH₂)ₙSi(CH₃)₃, or —(CH₂)ₙOSi(CH₃)ₘ, where each n is independently a value in the range of 1 to 4 inclusive, and each m is independently a value in the range of 0 to 4; and

wherein any of R¹³, R¹⁴, R¹⁵, R¹⁶, and R¹⁷ can together form a ring;

wherein X⁻ is an anion; and Cat is a cation that is in each instance independently selected from the group of cations as follows:

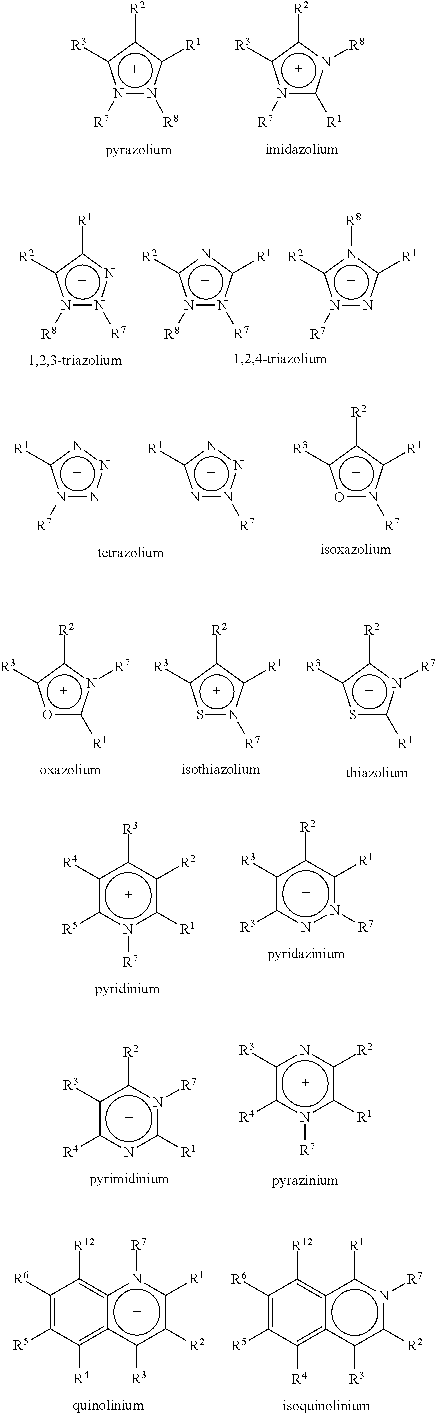

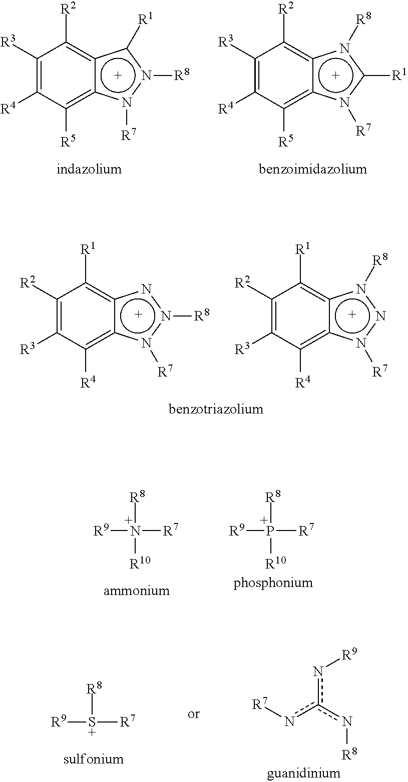

wherein in Cat (according to the formula for each of the members in the group of cations set forth above):

(a) R¹, R², R³, R⁴, R⁵, R⁶, and R¹² are independently selected from the group consisting of:

(i) H,

(ii) halogen such as Cl, Br, F, I,

(iii) a —CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH;

(iv) a —CH₃, —C₂H₅, or —C₃ to C₂₅ straight-chain, branched or cyclic alkane or alkene group comprising one to three heteroatoms selected from the group consisting of O, N, Si and S, and optionally substituted with at least one member selected from the group consisting of Cl, Br, F, I, OH, NH₂ and SH;

(v) a C₆ to C₂₀ unsubstituted aryl group, or a C₆ to C₂₅ unsubstituted heteroaryl group having one to three heteroatoms independently selected from the group consisting of O, N, Si and S;

(vi) a C₆ to C₂₅ substituted aryl group, or a C₆ to C₂₅ substituted heteroaryl group having one to three heteroatoms independently selected from the group consisting of O, N, Si and S; and wherein said substituted aryl or substituted heteroaryl group has one to three substituents independently selected from the group consisting of:
(A) a —CH₃, —C₂H₅, or C₃ to C₂₅ straight-chain, branched or cyclic alkane or alkene group, optionally substituted with at least one member selected from the group consisting of Cl, Br, F, I, OH, NH₂ and SH,
(B) OH,
(C) NH₂, and
(D) SH;

(vii) —(CH₂)ₙSi(CH₂)ₘCH₃, —(CH₂)ₙSi(CH₃)₃, or —(CH₂)ₙOSi(CH₃)ₘ, wherein each n is independently a value in the range of 1 to 4 and each m is independently a value in the range of 0 to 4;

(b) R⁷, R⁸, R⁹, and R¹⁰ is each independently selected from the group consisting of:

(i) a —CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH;

(ii) a —CH₃, —C₂H₅, or —C₃ to C₂₅ straight-chain, branched or cyclic alkane or alkene group comprising one to three heteroatoms selected from the group consisting of O, N, Si and S, and optionally substituted with at least one member selected from the group consisting of Cl, Br, F, I, OH, NH₂ and SH;

(iii) a C₆ to C₂₅ unsubstituted aryl group, or a C₆ to C₂₅ unsubstituted heteroaryl group having one to three heteroatoms independently selected from the group consisting of O, N, Si and S; and

(iv) a C₆ to C₂₅ substituted aryl group, or C₆ to C₂₅ substituted heteroaryl group having one to three heteroatoms independently selected from the group consisting of O, N, Si and S; and wherein said substituted aryl or substituted heteroaryl has one to three substituents independently selected from the group consisting of:
(A) a—CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH,
(B) OH,
(C) NH₂, and
(D) SH; (iv) —(CH₂)ₙSi(CH₂)ₘCH₃, —(CH₂)ₙSi(CH₃)₃, or —(CH₂)ₙOSi(CH₃)ₘ, where each n is independently a value in the range of 1 to 4, and each m is independently a value in the range of 0 to 4; and

(c) optionally at least two of R¹, R², R³, R⁴, R⁵, R⁶, R⁷, R⁸, R⁹, and R¹⁰ can together form a cyclic or bicyclic alkanyl or alkenyl group.

In one embodiment, in Formula 1 R¹³ is an electron rich (e.g. negatively charged) aryl group, R¹⁴ is a C₁˜C₁₀, or C₁˜C₈, or C₁˜C₆ alkyl group, and R¹⁵ is H or an electron withdrawing (electron deficient e.g. positively charged) group. In another embodiment in Formula II, III and Formula IV, R¹⁵ is H or an electron withdrawing group, and R¹³, R¹⁴, R¹⁶, and R¹⁷ are H, an alkyl group, or an aryl group.

In another embodiment, Cat comprises a cation selected from one or more members of the group consisting of pyridinium, pyridazinium, pyrimidinium, pyrazinium, imidazolium, pyrazolium, thiazolium, oxazolium, triazolium, phosphonium, ammonium, and guanidinium.

In another embodiment, X⁻ comprises an anion selected from one or more members of the group consisting of aminoacetate, ascorbate, benzoate, catecholate, citrate, dimethylphosphate, formate, fumarate, gallate, glycolate, glyoxylate, iminodiacetate, isobutyrate, kojate, lactate, levulinate, oxalate, pivalate, propionate, pyruvate, salicylate, succinamate, succinate, tiglate, tetrafluoroborate, tetrafluoroethanesulfonate, tropolonate, [CH₃CO₂]⁻, [HSO₄]⁻, [CH₃OSO₃], [C₂H₅OSO₃]⁻, [AlCl₄]⁻, [CO_{3]}²⁻, [HCO₃]⁻; [NO₂]⁻; [NO₃]⁻; [SO₄]²⁻, [PO₄]³⁻, [HPO₄]²⁻, [H₂PO₄], [HSO₃]⁻, [CuCl₂]⁻; Cl⁻, Br⁻, I⁻, SCN⁻, [BF₄]⁻, [PF₆]⁻, [SbF₆]⁻, [CF₃SO₃]³⁻, [HCF₂CF₂SO₃]⁻, [CF₃HFCCF₂SO₃]⁻, [HCClFCF₂SO₃]⁻, [(CF₃SO₂)₂N]⁻, [(CF₃CF₂SO₂)₂N]⁻, [(CF₃SO₂)₃C]⁻, [CF₃CO₂]⁻, [CF₃OCFHCF₂SO₃]⁻, [CF₃CF₂OCFHCF₂SO₃]⁻, [CF₃CFHOCF₂CF₂SO₃]⁻, [CF₂HCF₂OCF₂CF₂SO₃]⁻, [CF₂ICF₂OCF₂CF₂SO₃], [CF₃CF₂OCF₂CF₂SO₃]⁻, [(CF₂HCF₂SO₂)₂N]⁻, [(CF₃CFHCF₂SO₂)₂N]⁻, F⁻, and anions represented by the structure of the following formula:

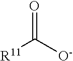

wherein R¹¹ is in each instance independently selected from the group consisting of:

(i) a —CH₃, —C₂H₅, or —C₃ to C₁₇ straight-chain, branched or cyclic alkane or alkene group, optionally substituted with at least one member selected from the group consisting of Cl, Br, F, I, OH, NH₂ and SH;

(ii) a —CH₃, —C₂H₅, or —C₃ to C₁₇ straight-chain, branched or cyclic alkane or alkene group comprising one to three heteroatoms selected from the group consisting of O, N, Si and S, and optionally substituted with at least one member selected from the group consisting of Cl, Br, F, I, OH, NH₂ and SH;

(iii) a C₆ to C₁₀ unsubstituted aryl group, or a C₆ to C₁₇ unsubstituted heteroaryl group having one to three heteroatoms independently selected from the group consisting of O, N, Si and S; and

(iv) a C₆ to C₁₀ substituted aryl group, or a C₃ to C₁₇ substituted heteroaryl group having one to three heteroatoms independently selected from the group consisting of O, N, Si and S; and wherein the substituted aryl or substituted heteroaryl group has one to three substituents independently selected from the group consisting of:
(A) a —CH₃, —C₂H₅, or —C₃ to C₁₇ straight-chain, branched or cyclic alkane or alkene, optionally substituted with at least one member selected from the group consisting of Cl, Br, F, I, OH, NH₂ and SH,
(B) OH,
(C) NH₂, and
(D) SH.

In another embodiment, X⁻ comprises an anion selected from one or more members of the group consisting of: [CH₃CO₂]⁻, [HSO₄]⁻, [CH₃OSO₃]⁻, [C₂H₅OSO₃]⁻, [AlCl₄]⁻, [CO₃]²⁻, [HCO₃]⁻, [NO₂]⁻, [NO₃]⁻, [SO₄]²⁻, [PO₃]³⁻, [HPO₃]²⁻, [H₂PO₃]¹⁻, [PO₄]³⁻, [HPO₄]²⁻, [H₂PO₄]⁻, [HSO₃]⁻, [CuCl₂]⁻, Cl⁻, Br⁻, I⁻, SCN⁻, and a fluorinated anion.

These compounds may by prepared, for example, from the amine-containing “task specific ionic liquids” (TSIL) described by Gutowski et al (J. Am. Chem. Soc. 130:14690-14704, 2008) and Davis et al (WO 2008/122030), and isothiocyanates according to the following reaction scheme:

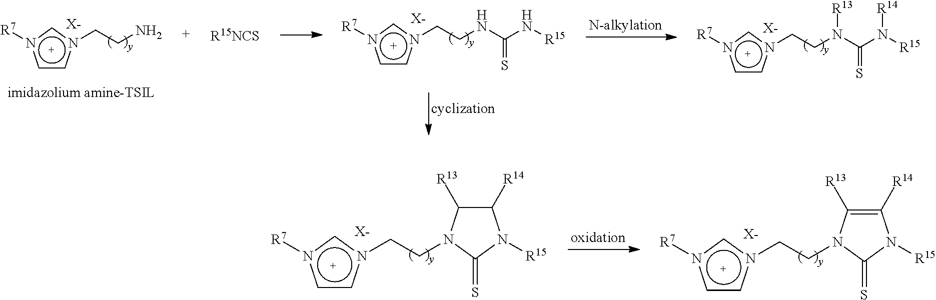

wherein X— represents any of the anions listed above, y is 0-15, and R⁷, R¹³, R¹⁴, and R¹⁵ are defined above. The thiourea ionic liquid adducts may be converted into heterocyclic thiones by cyclization.

A TSIL consisting of an immidazolium ion to which a primary amine moiety is covalently tethered was prepared by a process described in Bates et al, Volume 124, No. 6, 2002, Journal of the American Chemical Society, pages 926˜927 as follows: “The cation core is assembled by the reaction of 1-butylimidazole with 2-bromopropylamine hydrobromide in ethanol. After 24 hours under reflux, the ethanol is removed in vacuo, and the solid residue dissolved in a minimal quantity of water that is brought to ˜pH 8 by the addition, in small portions, of solid KOH. The product imidazolium bromide is then separated from the KBr byproduct by evaporation of the water, followed by extraction of the residue with ethanol-THF, in which the immidazolium salt is soluble. Subsequent ion-exchange with NaBF₄ in ethanol/water gives the product salt in 58% overall yield.”

The ionic compounds of Formulae I, II, III or IV are useful for the removal of CO₂ and/or SO₂ from a gaseous mixture in which they are contained. The gases and gaseous mixtures referred to herein may include vapors (volatilized liquids), gaseous compounds and/or other gaseous elements.

Also described herein are the compositions as represented by the structure of the following Formula IA, Formula IIA, Formula IIIA, or Formula IVA:

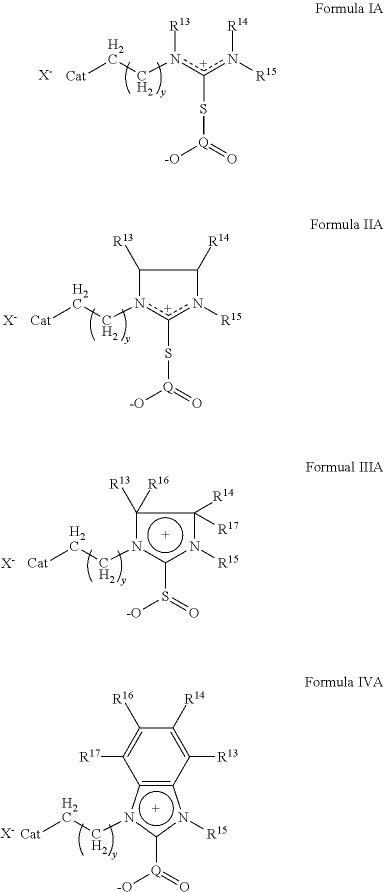

wherein Q is C or S, and all other variables are as set forth above with respect to the compounds of Formulae I˜IV.

In one embodiment, in Formula IA R¹³ is an electron rich (e.g. negatively charged) aryl group, R¹⁴ is a C₁˜C₁₀, or C₁˜C₈, or C₁˜C₆ alkyl group, and R¹⁵ is H or an electron withdrawing (electron deficient e.g. positively charged) group. In another embodiment in Formula IIA, IIIA and Formula IVA, R¹⁵ is H or an electron withdrawing group, and R¹³, R¹⁴, R¹⁶, and R¹⁷ are H, an alkyl group, or an aryl group.

The compositions of Formula IA, IIA, IIIA, and IVA can be prepared by the reaction of the ionic compounds of Formula I, II, III and IV with CO₂ and/or SO₂, as described herein. Contacting the compounds of Formula I, Formula II, Formula III, or Formula IV with a gaseous mixture containing CO₂ and/or SO₂ may be accomplished, for example, by any means that promotes intimate mixing of the compounds of Formula I with the source gas and is conducted for a time sufficient to allow significant removal of the targeted component(s). Thus, systems maximizing surface area contact are desirable. The conditions at which the process are conducted vary according to the compounds of the gaseous stream, the partial pressure of the CO₂ and/or SO₂, and equipment used, but in suitable embodiments be at temperatures ranging from ambient to about 200° C., and at pressures ranging from 1-5 atmospheres.

The gaseous mixture containing CO₂ and/or SO₂ can be any mixture of which CO₂ and/or SO₂ is a constituent part, or can be 100% CO₂, 100% SO₂, or any combination of CO₂ and SO₂. Examples of gaseous mixtures containing CO₂ and/or SO₂ include without limitation flue gases, combustion exhausts, natural gas streams, streams from rebreathing apparatus, and the products of chemical synthesis, degradation or fermentation operations.

Illustratively, contacting the compounds of Formula I, Formula II, Formula III, or Formula IV with a gaseous mixture can be performed by use of conventional liquid absorbers, such as counter-current liquid absorbers or cyclone scrubbers, by permeation through a supported liquid membrane, or by use of a fixed bed.

In one embodiment hereof, a liquid solvent can be used to remove a compound from a gas stream in an absorber, where gas and liquid are brought into contact countercurrently, and the gas is dissolved into the solvent. The absorber is typically equipped with trays or packing to provide a large liquid-gas contact area. Valve and sieve trays may be used, as may bubble cap and tunnel trays, where a tray typically has overflow weirs and downcomers to create hydrostatic holdup of the downward flow of the liquid. Random packings can also be used such as Rashig rings, Pall rings or Berl saddles, or structured packings of woven or nonwoven fabrics of metal, synthetic materials or ceramics.

The purified gas is taken off the head of the column. The solvent laden with the absorbed compound is withdrawn from the bottom of the absorber, routed to a regeneration system where it is freed of absorbed the absorbed gas component, and returned as lean solvent to the absorber. Regeneration may be accomplished by flash regeneration, which can involve pressure reduction and mild reboiling in one or more stages; by inert gas stripping; or by high temperature reboiling wherein the solvent is stripped by its own vapor, which is then condensed from the overhead gas and recycled as reflux.

In an absorber, a batch process may be performed where the flow rate through the vessel correlates to the residence time of contact and is suitably chosen to afford an effluent stream with the desired purification tolerance. To promote the desired intimate mixing, such gas/liquid absorption units also may be operated in a dual flow mode. Such dual flow can be co-current or counter-current. In such an embodiment, the gas mixture and the ionic liquid(s) flow through a purification unit contemporaneously. Methods for carbon dioxide absorption are further discussed in U.S. Pat. No. 6,579,343; US 2005/129,598; and US 2008/236,390 (each of which is by this reference incorporated as a part hereof for all purposes).

Where supported liquid membranes are used for gas recovery, the membrane may include a solvent such as an ionic liquid contained within the pores of a solid microporous support, such as a ceramic, metal, or polymeric support. Supported liquid membranes fabricated from supports such as ceramics, metals, and certain heat stable polymers may advantageously be used in higher than ambient temperature operations. Such higher temperature operations may be preferred to effect a more rapid separation, requiring less contact time. In addition, these higher temperature operations may also be a consequence of the process configuration, such as configurations requiring purification of high temperature exhaust gases or other gases exiting high temperature operations. Supported liquid membranes suitable for purifying high temperature gases obviate the need to pre-cool such gases before contact with the supported liquid membrane. The supported liquid membranes may be fabricated as thin films or hollow fibers with continuous networks of interconnected pores leading from one surface to the other. Supported liquid membranes contact a feed gas mixture on one side of the membrane and may effect separation of a gas component from the mixture by allowing that component to escape via permeation or diffusion into the compounds of Formula I, Formula II, Formula III, or Formula IV and through the liquid membrane.

The compounds of Formula I, Formula II, Formula III, or Formula IV can also be used in a conventional gas/liquid absorption unit-based system comprising a fixed bed. Such systems can be operated in batch mode or continuous flow mode. In a typical batch mode configuration, the compounds are introduced into a vessel followed by introduction of the gas mixture. After a prescribed residence time, the resulting gas is removed, leaving behind an impurity or group of impurities dissolved in the compounds of Formula I, Formula II, Formula III, or Formula IV. The batch purified gas can be generated by heating or reduced pressure treatment as described above. To maximize contact of compound and the gas mixture, the compounds of Formula I, Formula II, Formula III, or Formula IV can be coated on a solid support, such as glass beads, and the like, to increase the surface area of the compounds of Formula I, Formula II, Formula III, or Formula IV capable of contacting the gas mixture.

In one embodiment, this invention provides a method wherein the removal of CO₂ and/or SO₂ from a gaseous mixture occurs in a removal apparatus; wherein, in the removal apparatus, CO₂ and/or SO₂ is dissolved into compounds of Formula I, Formula II, Formula III, or Formula IV to form (i) a purified fraction that is depleted in CO₂ and/or SO₂ content (compared to the content thereof in the original feed of the gaseous mixture) and (ii) a solvent fraction that is enriched in CO₂ and/or SO₂ content (compared to the content thereof in the original feed of the gaseous mixture); and wherein the solvent fraction is separated from the removal apparatus. In a further alternative embodiment of the methods hereof, CO₂ and/or SO₂ can be separated from the solvent fraction to form a rectified solvent fraction, and the rectified solvent fraction can be returned to the removal apparatus.

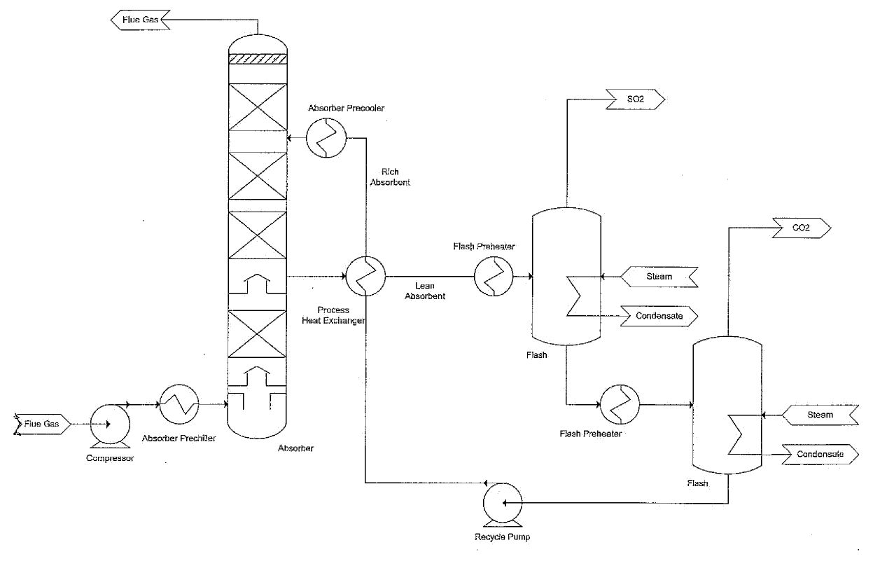

Equipment and processes that can be used for the absorption of CO₂ and/or SO₂ are further described in Absorption, Ullmann's Encyclopedia of Industrial Chemistry [2002, (Wiley-VCH Verlag GmbH & Co. KGa) Johann Schlauer and Manfred Kriebel, Jun. 15, 2000 (DOI: 10.1002/14356007.b03_{—}08)]; and Absorption, Kirk-Othmer Encyclopedia of Chemical Technology [2003, (John Wiley & Sons, Inc), Manuel Laso and Urs von Stockar (DOI:10.1002/0471238961.0102191519201503.a01.pub2)]. For example, one embodiment of an apparatus that can be used to selectively absorb and release CO₂ and SO₂ from the same gaseous mixture is shown in FIG. 1.

Without wishing to be bound by theory, it is believed that, when a gaseous mixture containing CO₂ and/or SO₂ are contacted with any one or more of the compounds of Formulae I˜IV, the CO₂ and SO₂ are separated from the gaseous mixture by binding to the nucleophilic compounds of Formula I, II and III to form a thiocarbonate or thiobisulfite, as illustrated below for compounds of Formula II and carbon dioxide:

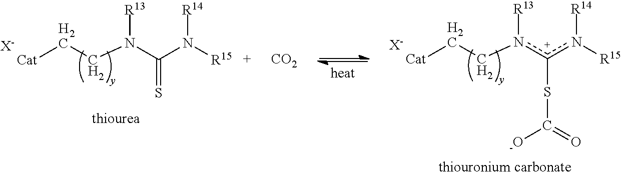

Upon heating, the carbon dioxide and sulfur dioxide can be released. One advantage of the present method is that due to the differences in bond strength, the sulfur dioxide and carbon dioxide can be released at different temperatures. This process is illustrated below:

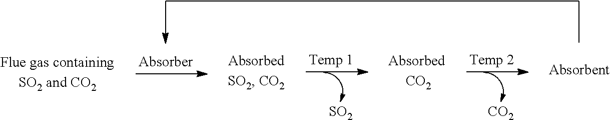

The compositions of Formula IA, IIA, IIIA and IVA find utility in their use, for example, as a source of either or both of (i) the ionic compounds of Formula I, II, III and IV, and (ii) CO₂ and SO₂. The compositions of Formula IA, IIA, IIIA, and IVA can be used in either of the following methods:

A method of generating an ionic compound as represented by the structure of the Formula I, II, III or IV, or a mixture thereof, by heating a composition as represented by the structure of the Formula IA, IIA, IIIA or IVA, or mixture thereof, and/or by contacting the composition as represented by the structure of the Formula IA, IIA, IIIA or IVA, or mixture thereof, with a non-solvent, and recovering the compound as represented by the structure of the Formula I, II, III or IV, or a mixture thereof.

A method of generating CO₂ and/or SO₂ by heating a composition as represented by the structure of the Formula IA, IIA, IIIA or IVA, or mixture thereof, and/or by contacting the composition as represented by the structure of the Formula IA, IIA, IIIA or IVA, or mixture thereof, with a non-solvent to separate CO₂ and/or SO₂ from the Formuale I˜IV compound, and recovering CO₂ and/or SO₂.

Other related systems, materials and methods for the removal of CO₂ or SO₂ from a gaseous mixture are disclosed in the following concurrently-filed U.S. provisional patent applications 61/313,298, 61/414,532, 61/416,421; 61/313,173; 61/313,181; 61/313,322; 61/313,328; 61/313,312; 61/313,183; and 61/313,191; each of which is by this reference incorporated in its entirety as a part hereof for all purposes.

Various materials suitable for use herein may be made by processes known in the art, and/or are available commercially from suppliers such as Alfa Aesar (Ward Hill, Mass.), City Chemical (West Haven, Conn.), Fisher Scientific (Fairlawn, N.J.), Sigma-Aldrich (St. Louis, Mo.) or Stanford Materials (Aliso Viejo, Calif.).

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value.

In this specification, unless explicitly stated otherwise or indicated to the contrary by the context of usage, where an embodiment of the subject matter hereof is stated or described as comprising, including, containing, having, being composed of or being constituted by or of certain features or elements, one or more features or elements in addition to those explicitly stated or described may be present in the embodiment. An alternative embodiment of the subject matter hereof, however, may be stated or described as consisting essentially of certain features or elements, in which embodiment features or elements that would materially alter the principle of operation or the distinguishing characteristics of the embodiment are not present therein. A further alternative embodiment of the subject matter hereof may be stated or described as consisting of certain features or elements, in which embodiment, or in insubstantial variations thereof, only the features or elements specifically stated or described are present.

In the various embodiments of this invention, an ionic compound formed by selecting any of the individual cations described or disclosed herein, and by selecting any of the individual anions described or disclosed herein, may be used for the purposes hereof. Correspondingly, in yet other embodiments, a subgroup of ionic liquids formed by selecting (i) a subgroup of any size of cations, taken from the total group of cations described and disclosed herein in all the various different combinations of the individual members of that total group, and (ii) a subgroup of any size of anions, taken from the total group of anions described and disclosed herein in all the various different combinations of the individual members of that total group, may be used for the purposes hereof. In forming an ionic compound, or a subgroup of ionic compounds, by making selections as aforesaid, the ionic compound or subgroup will be identified by, and used in, the absence of the members of the group of cations and/or the group of anions that are omitted from the total group thereof to make the selection; and, if desirable, the selection may thus be made in terms of the members of one or both of the total groups that are omitted from use rather than the members of the group(s) that are included for use.

Each of the formulae shown herein describes each and all of the separate, individual compounds and compositions that can be assembled in that formula by (1) selection from within the prescribed range for one of the variable radicals, substituents or numerical coefficents while all of the other variable radicals, substituents or numerical coefficents are held constant, and (2) performing in turn the same selection from within the prescribed range for each of the other variable radicals, substituents or numerical coefficents with the others being held constant. In addition to a selection made within the prescribed range for any of the variable radicals, substituents or numerical coefficents of only one of the members of the group described by the range, a plurality of compounds and compositions may be described by selecting more than one but less than all of the members of the whole group of radicals, substituents or numerical coefficents. When the selection made within the prescribed range for any of the variable radicals, substituents or numerical coefficents is a subgroup containing (i) only one of the members of the whole group described by the range, or (ii) more than one but less than all of the members of the whole group, the selected member(s) are selected by omitting those member(s) of the whole group that are not selected to form the subgroup. The compound, composition or plurality of compounds or compositions, may in such event be characterized by a definition of one or more of the variable radicals, substituents or numerical coefficents that refers to the whole group of the prescribed range for that variable but where the member(s) omitted to form the subgroup are absent from the whole group.

What is claimed is:

1. An ionic compound as represented by the structure of the following Formula I, Formula II, Formula III, or Formula IV: 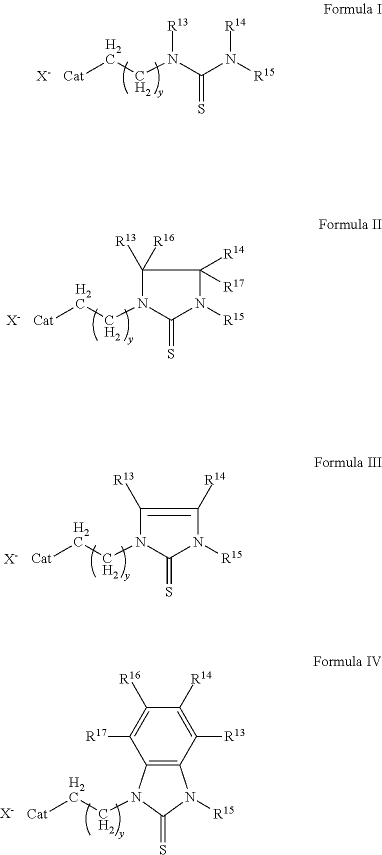
wherein y is a value in the range of 0 to 15 inclusive; and R¹³, R¹⁴, R¹⁵, R¹⁶, and R¹⁷ is each independently selected from the group consisting of:
(a) H,
(b) a —CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH;
(c) a —CH₃, —C₂H₅, or —C₃ to C₂₅ straight-chain, branched or cyclic alkane or alkene group comprising one to three heteroatoms selected from the group consisting of O, N, Si and S, and optionally substituted with at least one member selected from the group consisting of Cl, Br, F, I, OH, NH₂ and SH;
(d) a C₆ to C₂₀ unsubstituted aryl group, or a C₆ to C₂₅ unsubstituted heteroaryl group having one to three heteroatoms independently selected from the group consisting of O, N, Si and S;
(e) a C₆ to C₂₅ substituted aryl group, or a C₆ to C₂₅ substituted heteroaryl group having one to three heteroatoms independently selected from the group consisting of O, N, Si and S; wherein the substituted aryl or substituted heteroaryl group has one to three substituents independently selected from the group consisting of:
(i) a —CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH,
(ii) OH,
(iii) NH₂, and
(iv) SH; and
(f) —(CH₂)ₙSi(CH₂)ₘCH₃, —(CH₂)ₙSi(CH₃)₃, or —(CH₂)ₙOSi(CH₃)ₘ, wherein each n is independently a value in the range of 1 to 4 inclusive, and each m is independently a value in the range of 0 to 4; and
wherein any of R¹³, R¹⁴, R¹⁵, R¹⁶, and R¹⁷ can together form a ring;
wherein X⁻ is an anion; and Cat is a cation that is in each instance independently selected from the group of cations as follows: 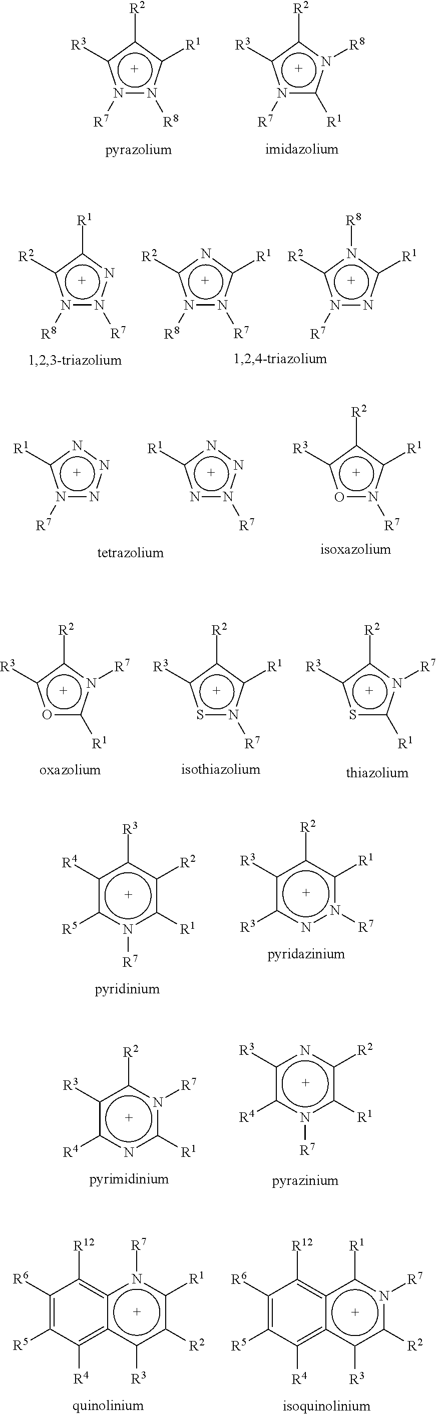 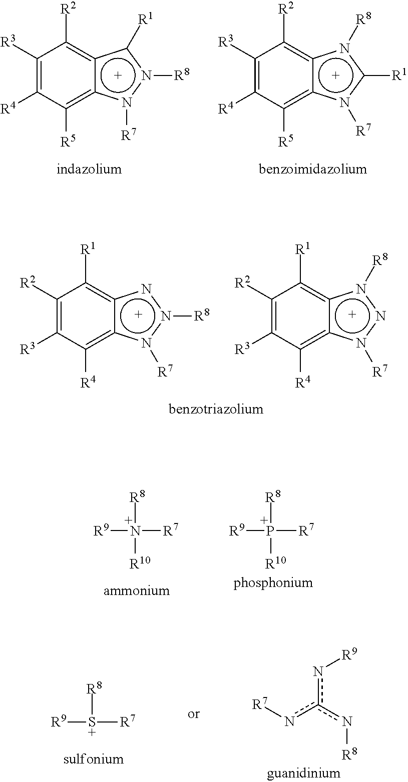
wherein in Cat (according to the formula for each of the members in the group of cations set forth above):
(a) R¹, R², R³, R⁴, R⁵, R⁶, and R¹² are independently selected from the group consisting of:
(i) H,
(ii) halogen,
(iii) a —CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH;
(iv) a —CH₃, —C₂H₅, or —C₃ to C₂₅ straight-chain, branched or cyclic alkane or alkene group comprising one to three heteroatoms selected from the group consisting of O, N, Si and S, and optionally substituted with at least one member selected from the group consisting of Cl, Br, F, I, OH, NH₂ and SH;
(v) a C₆ to C₂₀ unsubstituted aryl group, or a C₆ to C₂₅ unsubstituted heteroaryl group having one to three heteroatoms independently selected from the group consisting of O, N, Si and S;
(vi) a C₆ to C₂₅ substituted aryl group, or a C₆ to C₂₅ substituted heteroaryl group having one to three heteroatoms independently selected from the group consisting of O, N, Si and S; and wherein said substituted aryl or substituted heteroaryl group has one to three substituents independently selected from the group consisting of:
(A) a —CH₃, —C₂H₅, or C₃ to C₂₅ straight-chain, branched or cyclic alkane or alkene group, optionally substituted with at least one member selected from the group consisting of Cl, Br, F, I, OH, NH₂ and SH,
(B) OH,
(C) NH₂, and
(D) SH;
(vii) —(CH_{2l )}ₙSi(CH₂)ₘCH₃, —(CH₂)ₙSi(CH₃)₃, or —(CH₂)ₙOSi(CH₃)ₘ, wherein each n is independently a value in the range of 1 to 4 and each m is independently a value in the range of 0 to 4;
(b) R⁷, R⁸, R⁹, and R¹⁰ is each independently selected from the group consisting of:
(i) a —CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH;
(ii) a —CH₃, —C₂H₅, or —C₃ to C₂₅ straight-chain, branched or cyclic alkane or alkene group comprising one to three heteroatoms selected from the group consisting of O, N, Si and S, and optionally substituted with at least one member selected from the group consisting of Cl, Br, F, I, OH, NH₂ and SH;
(iii) a C₆ to C₂₅ unsubstituted aryl group, or a C₆ to C₂₅ unsubstituted heteroaryl group having one to three heteroatoms independently selected from the group consisting of O, N, Si and S; and
(iv) a C₆ to C₂₅ substituted aryl group, or C₆ to C₂₅ substituted heteroaryl group having one to three heteroatoms independently selected from the group consisting of O, N, Si and S; and wherein said substituted aryl or substituted heteroaryl has one to three substituents independently selected from the group consisting of:
(A) a—CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH,
(B) OH,
(C) NH₂, and
(D) SH;
(iv) —(CH₂)ₙSi(CH₂)ₘCH₃, —(CH₂)ₙSi(CH₃)₃, or —(CH₂)ₙOSi(CH₃)ₘ, where each n is independently a value in the range of 1 to 4, and each m is independently a value in the range of 0 to 4; and
(c) optionally at least two of R¹, R², R³, R⁴, R⁵, R^{6,} R⁷, R⁸, R⁹, and R¹⁰ can together form a cyclic or bicyclic alkanyl or alkenyl group.

2. The ionic compound of claim 1 wherein, in Formula I, R¹⁴ is a C₁˜C₁₀ alkyl group.

3. The ionic compound of claim 1 wherein in Formula II, III and Formula IV, R¹⁵ is H, and R¹³, R¹⁴, R¹⁶, and R¹⁷ are H, an alkyl group, or an aryl group.

4. The ionic compound of claim 1 wherein X⁻ is selected from one or more members of the group consisting of: aminoacetate, ascorbate, benzoate, catecholate, citrate, dimethylphosphate, formate, fumarate, gallate, glycolate, glyoxylate, iminodiacetate, isobutyrate, kojate, lactate, levulinate, oxalate, pivalate, propionate, pyruvate, salicylate, succinamate, succinate, tiglate, tetrafluoroborate, tetrafluoroethanesulfonate, tropolonate, [CH₃CO₂]⁻, [HSO₄]⁻, [CH₃OSO₃], [C₂H₅OSO₃]⁻, [AlCl₄]⁻, [CO_{3]}²⁻, [HCO₃]⁻; [NO₂]⁻; [NO₃]⁻; [SO₄]²⁻, [PO₄]³⁻, [HPO₄]²⁻, [H₂PO₄], [HSO₃]⁻, [CuCl₂]⁻; Cl⁻, Br⁻, I⁻, SCN⁻, [BF₄]⁻, [PF₆]⁻, [SbF₆]⁻, [CF₃SO₃]³⁻, [HCF₂CF₂SO₃]⁻, [CF₃HFCCF₂SO₃]⁻, [HCClFCF₂SO₃]⁻, [(CF₃SO₂)₂N]⁻, [(CF₃CF₂SO₂)₂N]⁻, [(CF₃SO₂)₃C]⁻, [CF₃CO₂]⁻, [CF₃OCFHCF₂SO₃]⁻, [CF₃CF₂OCFHCF₂SO₃]⁻, [CF₃CFHOCF₂CF₂SO₃]⁻, [CF₂HCF₂OCF₂CF₂SO₃]⁻, [CF₂ICF₂OCF₂CF₂SO₃], [CF₃CF₂OCF₂CF₂SO₃]⁻, [(CF₂HCF₂SO₂)₂N]⁻, [(CF₃CFHCF₂SO₂)₂N]⁻, F⁻, and anions represented by the structure of the following formula: 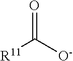
wherein R¹¹ is in each instance independently selected from the group consisting of:
(i) a —CH₃, —C₂H₅, or —C₃ to C₁₇ straight-chain, branched or cyclic alkane or alkene group, optionally substituted with at least one member selected from the group consisting of Cl, Br, F, I, OH, NH₂ and SH;
(ii) a —CH₃, —C₂H₅, or —C₃ to C₁₇ straight-chain, branched or cyclic alkane or alkene group comprising one to three heteroatoms selected from the group consisting of O, N, Si and S, and optionally substituted with at least one member selected from the group consisting of Cl, Br, F, I, OH, NH₂ and SH;
(iii) a C₆ to C₁₀ unsubstituted aryl group, or a C₆ to C₁₇ unsubstituted heteroaryl group having one to three heteroatoms independently selected from the group consisting of O, N, Si and S; and
(iv) a C₆ to C₁₀ substituted aryl group, or a C₃ to C₁₇ substituted heteroaryl group having one to three heteroatoms independently selected from the group consisting of O, N, Si and S; and wherein the substituted aryl or substituted heteroaryl group has one to three substituents independently selected from the group consisting of:
(A) a —CH₃, —C₂H₅, or —C₃ to C₁₇ straight-chain, branched or cyclic alkane or alkene, optionally substituted with at least one member selected from the group consisting of Cl, Br, F, I, OH, NH₂ and SH,
(B) OH,
(C) NH₂, and
(D) SH.

5. A composition as represented by the structure of the following Formula IA, Formula IIA, Formula IIIA, or Formula IVA: 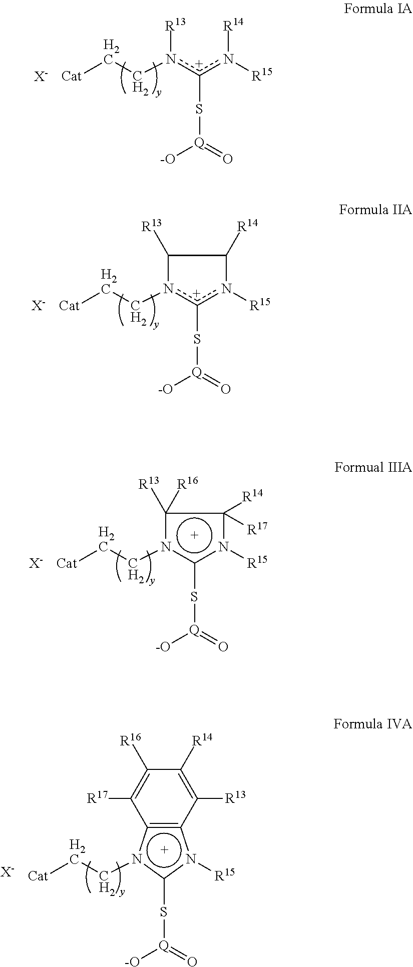
wherein Q is C or S; y is a value in the range of 0 to 15 inclusive; and R¹³, R¹⁴, R¹⁵, R¹⁶, and R¹⁷ is each independently selected from the group consisting of:
(a) H,
(b) a —CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH;
(c) a —CH₃, —C₂H₅, or —C₃ to C₂₅ straight-chain, branched or cyclic alkane or alkene group comprising one to three heteroatoms selected from the group consisting of O, N, Si and S, and optionally substituted with at least one member selected from the group consisting of Cl, Br, F, I, OH, NH₂ and SH;
(d) a C₆ to C₂₀ unsubstituted aryl group, or a C₆ to C₂₅ unsubstituted heteroaryl group having one to three heteroatoms independently selected from the group consisting of O, N, Si and S;
(e) a C₆ to C₂₅ substituted aryl group, or a C₆ to C₂₅ substituted heteroaryl group having one to three heteroatoms independently selected from the group consisting of O, N, Si and S; wherein the substituted aryl or substituted heteroaryl group has one to three substituents independently selected from the group consisting of:
(i) a —CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH,
(ii) OH,
(iii) NH₂, and
(iv) SH; and
(f) —(CH₂)ₙSi(CH₂)ₘCH₃, —(CH₂)ₙSi(CH₃)₃, or —(CH₂)ₙOSi(CH₃)ₘ, where each n is independently a value in the range of 1 to 4 inclusive, and each m is independently a value in the range of 0 to 4; and
wherein any of R¹³, R¹⁴, R¹⁵, R¹⁶, and R¹⁷ can together form a ring;
wherein X⁻ is an anion; and Cat is a cation that is in each instance independently selected from the group of cations as follows: 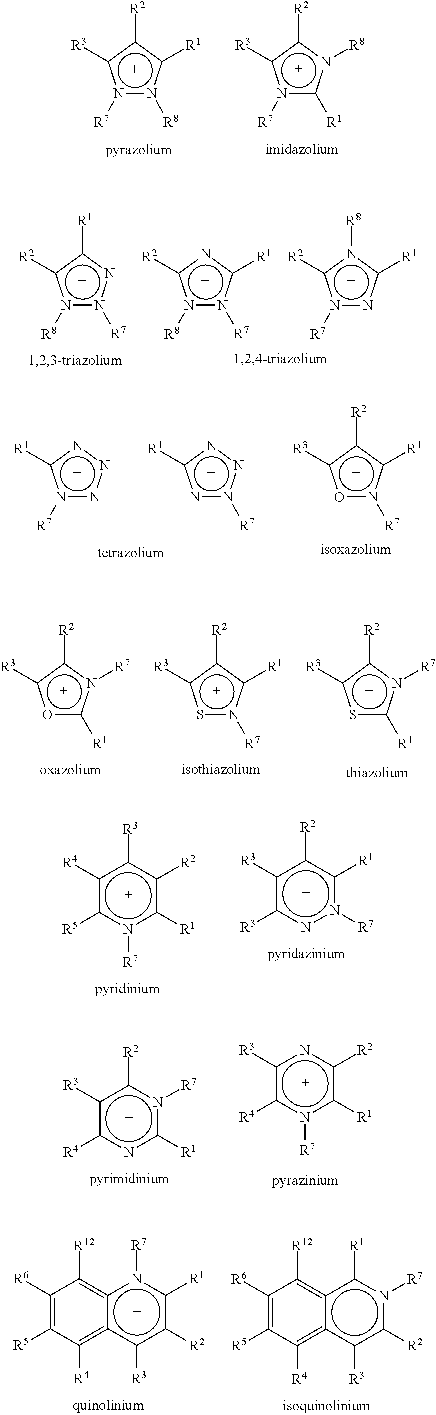 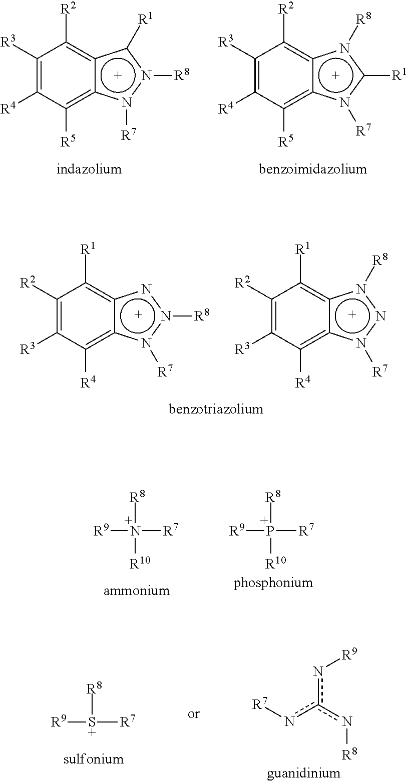
wherein in Cat (according to the formula for each of the members in the group of cations set forth above):
(a) R¹, R², R³, R⁴, R⁵, R⁶, and R¹² are independently selected from the group consisting of:
(i) H,
(ii) halogen,
(iii) a —CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH;
(iv) a —CH₃, —C₂H₅, or —C₃ to C₂₅ straight-chain, branched or cyclic alkane or alkene group comprising one to three heteroatoms selected from the group consisting of O, N, Si and S, and optionally substituted with at least one member selected from the group consisting of Cl, Br, F, I, OH, NH₂ and SH;
(v) a C₆ to C₂₀ unsubstituted aryl group, or a C₆ to C₂₅ unsubstituted heteroaryl group having one to three heteroatoms independently selected from the group consisting of O, N, Si and S;
(vi) a C₆ to C₂₅ substituted aryl group, or a C₆ to C₂₅ substituted heteroaryl group having one to three heteroatoms independently selected from the group consisting of O, N, Si and S; and wherein said substituted aryl or substituted heteroaryl group has one to three substituents independently selected from the group consisting of:
(A) a —CH₃, —C₂H₅, or C₃ to C₂₅ straight-chain, branched or cyclic alkane or alkene group, optionally substituted with at least one member selected from the group consisting of Cl, Br, F, I, OH, NH₂ and SH,
(B) OH,
(C) NH₂, and
(D) SH;
(vii) —(CH₂)ₙSi(CH₂)ₘCH₃, —(CH₂)ₙSi(CH₃)₃, or —(CH₂)ₙOSi(CH₃)ₘ, wherein each n is independently a value in the range of 1 to 4 and each m is independently a value in the range of 0 to 4;
(b) R⁷, R⁸, R⁹, and R¹⁰ is each independently selected from the group consisting of:
(i) a —CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH;
(ii) a —CH₃, —C₂H₅, or —C₃ to C₂₅ straight-chain, branched or cyclic alkane or alkene group comprising one to three heteroatoms selected from the group consisting of O, N, Si and S, and optionally substituted with at least one member selected from the group consisting of Cl, Br, F, I, OH, NH₂ and SH;
(iii) a C₆ to C₂₅ unsubstituted aryl group, or a C₆ to C₂₅ unsubstituted heteroaryl group having one to three heteroatoms independently selected from the group consisting of O, N, Si and S; and
(iv) a C₆ to C₂₅ substituted aryl group, or C₆ to C₂₅ substituted heteroaryl group having one to three heteroatoms independently selected from the group consisting of O, N, Si and S; and wherein said substituted aryl or substituted heteroaryl has one to three substituents independently selected from the group consisting of:
(A) a—CH₃, —C₂H₅, or —C₃ to C₂₅ straight-chain, branched or cyclic alkane or alkene group, optionally substituted with at least one member selected from the group consisting of Cl, Br, F, I, OH, NH₂ and SH,
(B) OH,
(C) NH₂, and
(D) SH;
(iv) —(CH₂)ₙSi(CH₂)ₘCH₃, —(CH₂)ₙSi(CH₃)₃, or —(CH₂)ₙOSi(CH₃)ₘ, where each n is independently a value in the range of 1 to 4, and each m is independently a value in the range of 0 to 4; and
(c) optionally at least two of R¹, R², R³, R⁴, R⁵, R⁶, R⁷, R⁸, R⁹, and R¹⁰ can together form a cyclic or bicyclic alkanyl or alkenyl group.

6. The composition of claim 5 wherein, in Formula I, R¹⁴ is a C₁˜C₁₀ alkyl group.

7. The composition of claim 5 wherein in Formula II, III and Formula IV, R¹⁵ is H, and R¹³, R¹⁴; R¹⁶, and R¹⁷ are H, an alkyl group, or an aryl group.

8. The composition of claim 5 wherein X⁻ is selected from one or more members of the group consisting of: aminoacetate, ascorbate, benzoate, catecholate, citrate, dimethylphosphate, formate, fumarate, gallate, glycolate, glyoxylate, iminodiacetate, isobutyrate, kojate, lactate, levulinate, oxalate, pivalate, propionate, pyruvate, salicylate, succinamate, succinate, tiglate, tetrafluoroborate, tetrafluoroethanesulfonate, tropolonate, [CH₃CO₂]⁻, [HSO₄]⁻, [CH₃OSO₃], [C₂H₅OSO₃]⁻, [AlCl₄]⁻, [CO_{3]}²⁻, [HCO₃]⁻; [NO₂]⁻; [NO₃]⁻; [SO₄]²⁻, [PO₄]³⁻, [HPO₄]²⁻, [H₂PO₄], [HSO₃]⁻, [CuCl₂]⁻; Cl⁻, Br⁻, I⁻, SCN⁻, [BF₄]⁻, [PF₆]⁻, [SbF₆]⁻, [CF₃SO₃]³⁻, [HCF₂CF₂SO₃]⁻, [CF₃HFCCF₂SO₃]⁻, [HCClFCF₂SO₃]⁻, [(CF₃SO₂)₂N]⁻, [(CF₃CF₂SO₂)₂N]⁻, [(CF₃SO₂)₃C]⁻, [CF₃CO₂]⁻, [CF₃OCFHCF₂SO₃]⁻, [CF₃CF₂OCFHCF₂SO₃]⁻, [CF₃CFHOCF₂CF₂SO₃]⁻, [CF₂HCF₂OCF₂CF₂SO₃]⁻, [CF₂ICF₂OCF₂CF₂SO₃], [CF₃CF₂OCF₂CF₂SO₃]⁻, [(CF₂HCF₂SO₂)₂N]⁻, [(CF₃CFHCF₂SO₂)₂N]⁻, F⁻, and anions represented by the structure of the following formula: 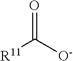
wherein R¹¹ is in each instance independently selected from the group consisting of:
(i) a —CH₃, —C₂H₅, or —C₃ to C₁₇ straight-chain, branched or cyclic alkane or alkene group, optionally substituted with at least one member selected from the group consisting of Cl, Br, F, I, OH, NH₂ and SH;
(ii) a —CH₃, —C₂H₅, or —C₃ to C₁₇ straight-chain, branched or cyclic alkane or alkene group comprising one to three heteroatoms selected from the group consisting of O, N, Si and 5, and optionally substituted with at least one member selected from the group consisting of Cl, Br, F, I, OH, NH₂ and SH;
(iii) a C₆ to C₁₀ unsubstituted aryl group, or a C₆ to C₁₇ unsubstituted heteroaryl group having one to three heteroatoms independently selected from the group consisting of O, N, Si and S; and
(iv) a C₆ to C10 substituted aryl group, or a C₃ to C₁₇ substituted heteroaryl group having one to three heteroatoms independently selected from the group consisting of O, N, Si and S; and wherein the substituted aryl or substituted heteroaryl group has one to three substituents independently selected from the group consisting of:
(A) a —CH₃, —C₂H₅, or —C₃ to C₁₇ straight-chain, branched or cyclic alkane or alkene, optionally substituted with at least one member selected from the group consisting of Cl, Br, F, I, OH, NH₂ and SH,
(B) OH,
(C) NH₂, and
(D) SH.

9. The composition of claim 5 wherein Q is C.

10. A method of generating an ionic compound as represented by the structure of the Formula I, II, III or IV in claim 1, or a mixture thereof, by heating a composition as represented by the structure of the Formula IA, IIA, IIIA or IVA in claim 5, or mixture thereof, and/or by contacting the composition as represented by the structure of the Formula IA, IIA, IIIA or IVA in claim 5, or mixture thereof, with a non-solvent, and recovering the compound as represented by the structure of the Formula I, II, III or IV in claim 1, or a mixture thereof.

11. A method of generating CO₂ and/or SO₂ by heating a composition as represented by the structure of the Formula IA, IIA, IIIA or IVA in claim 5, or mixture thereof, and/or by contacting the composition as represented by the structure of the Formula IA, IIA, IIIA or IVA in claim 5, or mixture thereof, with a non-solvent, and recovering CO₂ and/or SO₂.
